(19) United States
(12) Patent Application Publication
(10) Pub. No.: US 2026/0308845 A1
(43) Pub. Date: Oct. 8, 2026

(54) BIOMASS PROCESSING SYSTEM FOR TREATMENT OF BIOMASS MATERIAL

(71) Applicant: VALMET AB, Sundsvall (SE)

(72) Inventor: Christer HÄGGLUND, Bergeforsen (SE)

(73) Assignee: VALMET AB, Sundsvall (SE)

(21) Appl. No.: 19/482,040

(22) PCT Filed: May 14, 2024
(86) PCT No.: PCT/SE2024/050460

§ 371 Date: Nov. 6, 2025

(30) Foreign Application Priority Data
May 23, 2023 (SE) 2330234-2

(51) Int. Cl. D21C 1/02; B01J 3/02; B01J 3/04; F16K 3/03

(52) U.S. Cl. CPC D21C 1/02; B01J 3/02; B01J 3/04; F16K 3/03

(57) ABSTRACT

A blow valve 15 and a biomass processing system 1 comprising such a blow valve Said blow valve 15 being connectable in a blow line 7 which is connecting two differently pressurized vessels 3, 5 of the biomass processing system 1. Said blow valve 15 is configured for controlling the flow of biomass material and fluid in the blow line 7, wherein said blow valve 15 comprises a shutter valve 31 with a central opening 33 of controllable size, said shutter valve 31 comprising a number of valve plates 35 which are disposed in an annular pattern surrounding the central opening 33 of the shutter valve 31 and wherein said valve plates 35 are movable in relation to each other whereby the size of the central opening 33 is controlled by moving the valve plates 35.

### TECHNICAL FIELD OF THE INVENTION

The present invention relates to a biomass processing system.

### BACKGROUND

Systems for pretreatment or prehydrolysis of biomass are known in the art. Such biomass processing systems may comprise one or more pressurized vessels in which the biomass is pretreated. The pressurized vessel can for example be a refiner or a reactor. In some systems the biomass is treated in the pressurized vessel with steam at elevated pressure and temperature with or without the addition of chemicals.

Typically, biomass material is treated with steam under pressure in a reactor and then transferred to a steam separation device, e.g. a cyclone, through a transporting pipe, often called a blow line. The steam separation device can be pressurized or working at or below atmospheric pressure. However, the steam separation device will be at a lower pressure than the reactor. In the example of pulp production, the pressurized vessel may be a refiner and biomass material is discharged from the refiner via the blow line to another vessel, typically held at atmospheric pressure. A valve, often called a blow valve, is usually provided close to the outlet from the reactor/refiner in the blow line. Said blow valve is provided for controlling the flow through the blow line. The blow valve will hereby provide a pressure drop to the biomass which is transported through the blow line out from the reactor/refiner. There are several established ways of discharging material from pretreatment vessels, e.g. steam explosion (steam and moist particles in the flow) and dilution discharge (liquid and particles in the flow) or combinations of these two methods. The highest velocity in the blow line is typically obtained with a steam explosion discharge. Particles (biomass, sand or other impurities) which are travelling with high speed in the blow line will cause the blow line to erode. Erosion of the blow line is a big problem. Blow lines need to be changed or repaired frequently due to this. Especially directly after the blow valve there are often problems with erosion due to particles impinging the blow line surface unevenly, i.e. due to the valve construction. Particles will be distributed unevenly over the blow line circumference and particles will impinge certain points of the blow line surface more which will lead to erosion problems. At bends/elbows/junctions of the blow line the erosion problem is usually also big. A common way to handle these erosion problems is to minimize the number of bends, to have large radius on the bends or to use wear resistant liners in the bends and close to the blow valve.

### SUMMARY

An object of the invention is to provide an improved biomass processing system.

This is achieved by a biomass processing system according to the independent claim.

According to the invention, a biomass processing system for treatment of biomass material is provided, wherein said biomass processing system is a biomass conversion system, said biomass processing system comprises:
a first vessel in which said biomass material is treated under a first pressure;
a second vessel in which said biomass material is received and held at a second pressure which is lower than the first pressure, wherein the difference between the first and second pressures is at least 10 bar;
a blow line connecting an outlet of the first vessel with an inlet of the second vessel for transporting the biomass material from the first vessel to the second vessel; and
a blow valve being connected in said blow line and being configured for controlling the flow of biomass material and fluid in the blow line and configured to control a steam explosion of the material occurring at the passage through the blow valve, wherein said blow valve comprises a shutter valve with a central opening of controllable size, said shutter valve comprising a number of valve plates which are disposed in an annular pattern surrounding the central opening of the shutter valve and wherein said valve plates are movable in relation to each other whereby the size of the central opening is controlled by moving the valve plates.

In one embodiment of the invention, the blow valve is designed with dimensions and materials for being usable and approved for pressures up to at least 25 bar(g).

The biomass processing system may be a biomass conversion system, for example for production of sugars, bioethanol, biogas, biochar, steam exploded pellets or various chemicals.

In one embodiment of the invention, the shutter valve comprises between 4 and 20 or between 6 and 16 valve plates.

In one embodiment of the invention, said valve plates have a thickness of between 10 mm and 40 mm. Hereby they can withstand high pressures.

In one embodiment of the invention, said valve plates are made from a high yield steel material. Hereby they are wear resistant.

In one embodiment of the invention, said valve plates are coated with a wear resistant lining.

In one embodiment of the invention, said valve plates have a form of a wedge having two straight side edges which meet in a tip of the wedge.

In one embodiment of the invention, the blow valve further comprises a wear lining. Hereby a life time of the blow valve can be extended thanks to the wear lining protecting an inner surface of the blow valve. Furthermore, the wear lining can be exchanged to a new wear lining when the wear lining has been worn out.

In one embodiment of the invention, said blow valve comprises at least one sealing positioned in the blow valve such that leakage of biomass material and fluid via the blow valve outside said central opening is limited.

In one embodiment of the invention, the blow valve is dimensioned to fit in a blow line having a diameter between 50-150 mm.

In one embodiment of the invention, the difference between the first and second pressures of the first and second vessels is at least 10 bar.

In one embodiment of the invention, said biomass processing system is a biomass conversion system, for example for production of sugars, bioethanol, biogas, biochar, steam exploded pellets or various chemicals.

In one embodiment of the invention, the blow valve is connected directly to the outlet of the first vessel.

In one embodiment of the invention, the blow line has a diameter between 50-150 mm.

In one embodiment of the invention, said vessel is a first vessel being a reactor in a biomass conversion system.

### BRIEF DESCRIPTION OF THE DRAWINGS

FIG. 1 shows schematically a biomass processing system according to one embodiment of the invention.

FIGS. 2a-2d show a blow valve in four different positions.

FIG. 3 is a perspective view of a part of the blow valve as shown in FIGS. 2a-2d.

FIG. 4 shows the same part of the blow valve as shown in FIG. 3 with an added valve plate movement control disc.

FIG. 5 shows in perspective view a part of the blow valve as shown in FIGS. 2a-2d.

FIG. 6 is a cross section in perspective of the blow valve as shown in FIGS. 2a-2d.

FIG. 7 is a cross section of a blow valve.

### DETAILED DESCRIPTION OF EMBODIMENTS

The invention relates to biomass processing system. A biomass processing system may be a cellulose pulp production system comprising a refiner in which the biomass material is treated. A biomass processing system may also be a system where biomass material is treated by steam at elevated pressure and temperature in a reactor as a step in a process for production of for example sugars, bioethanol, biogas, biochar, steam exploded pellets or various chemicals. The biomass material can be for example wood in different forms (wood chips, saw dust, splinters, etc), wheat straw, rice straw, bagasse, corn stover, empty fruit bunches, energy cane, different kinds of grass material or similar biomass materials.

Examples of treatment of biomass are auto or acid hydrolysis followed by steam explosion discharge, dilution discharge or combinations of these as described in the background. These methods differ by the content of liquid/water in the biomass material.

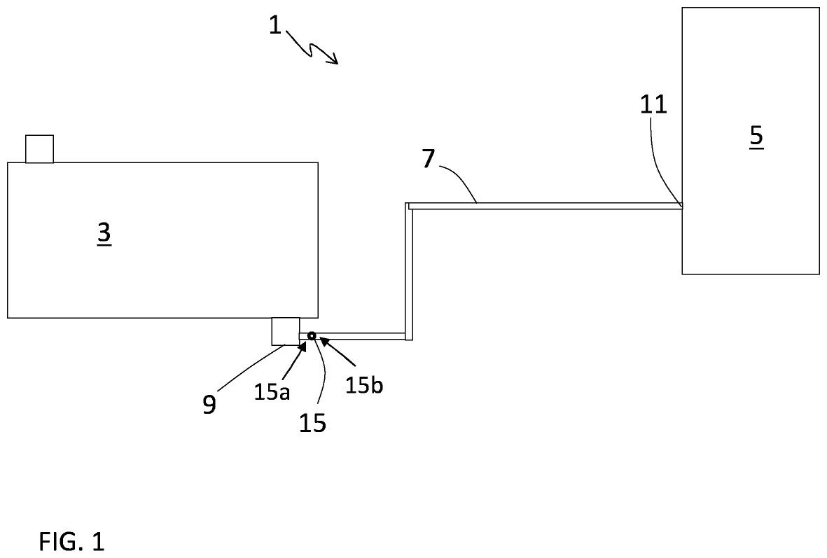

In FIG. 1 a biomass processing system 1 for treatment of biomass material according to one embodiment of the invention is shown schematically. Said biomass processing system 1 comprises a first vessel 3 in which said biomass material is treated at a first pressure. Said first vessel can for example be a reactor or a refiner and can be used for pretreatment of biomass material at elevated pressure and temperature for example by steam. If the first vessel is a refiner, it is usually connected to a reactor at an inlet of the refiner. The first vessel 3 may comprise a discharge device provided at an outlet 9 of the first vessel (The outlet 9 of the first vessel 3 is schematically shown and may comprise a discharge device). The pressure in the first vessel 3 can typically be in the range 5-30 bar(g), but significantly higher pressures are also used in these systems. The temperature in the first vessel 3 can typically be in the range of 130-250° C. The system comprises further a second vessel 5 in which said biomass material is collected at a second pressure which is lower than the first pressure. The second vessel can be a collection vessel at atmospheric pressure or in some examples a separation device, such as a gas separation device which is separating gas from solids and/or liquid, e.g. a cyclone or a mechanical separator. The second vessel 5 can be a pressurized vessel or at atmospheric pressure. The biomass processing system 1 comprises furthermore a blow line 7 connecting the outlet 9 of the first vessel 3 with an inlet 11 of the second vessel 5 for transporting the biomass material from the first vessel 3 to the second vessel 5.

Usually a valve 15, often called a blow valve 15, is provided in the blow line 7 close to the outlet 9 from the first vessel 3 or directly connected to the outlet 9 of the first vessel 3. The blow valve 15 is provided for controlling the flow of biomass material and steam in the blow line 7. The blow valve 15 is also used to control a possible steam explosion of the material occurring at the passage through the blow valve 15. The steam explosion is a part of the treatment of the biomass material. Furthermore, a steam consumption is also controlled by controlling the opening size of the blow valve. Hereby a blow valve 15 provided in a biomass processing system is not only used for controlling a flow but is instead an active part of the biomass treatment and an important part of the system which needs to be specifically designed for this function, these pressures and to withstand erosion.

Commonly used blow valves in these types of biomass processing systems are for example ball valves and piston valves, which valves force the flow of biomass material towards one side of a flow channel of the valve. An example of such a valve is EvoThrottle™ from Valmet. This non-symmetric distribution of the flow inside a flow channel of the valve will cause erosion problems.

There are problems with erosion in the blow valve both in pulp production systems and in other biomass conversion systems, where for example biogas, ethanol, biochar and biochemicals are produced as discussed above. However, the erosion problems may be most problematic in biomass conversion systems where pressures are higher and where the biomass has a lower moisture content than in pulp production systems. Particles, such as sand which will increase erosion further, may be more frequent in lignocellulosic raw material which is used in some of the biomass conversion systems. A blow valve will be described below, which will decrease erosion problems in the blow valve itself and in the blow line connected to the blow valve will be especially suitable in a biomass conversion system in which for example biogas, ethanol, biochar and biochemicals are produced.

A blow valve 15 for use in a biomass processing system 1 as schematically shown in FIG. 1 and described above is provided. The blow valve 15 is connectable in a blow line 7 which is connecting a first vessel 3 and a second vessel 5, which are two differently pressurized vessels of the biomass processing system 1 and it is configured for controlling the flow of biomass material and fluid in the blow line 7. An inlet end 15a of the blow valve 15 may be connected directly to the outlet 9 of the first vessel 3 and an outlet end 15b of the valve 15 is then connected to the blow line 7. In other examples the blow valve 15 may be connected in the blow line 7 at a distance from the first vessel 3, however the blow valve 15 is suitably provided close to the first vessel 3, i.e. closer to the first vessel 3 than to the second vessel 5.

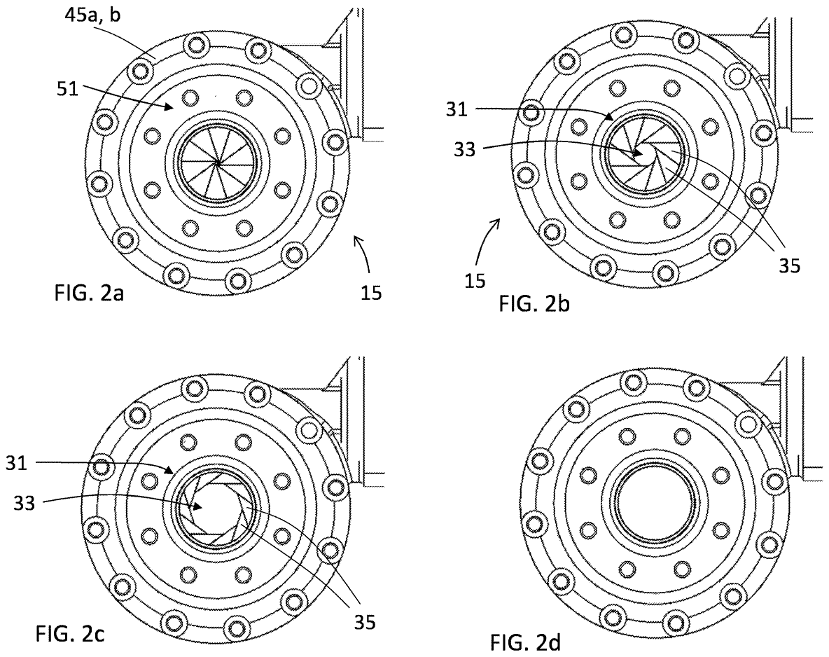

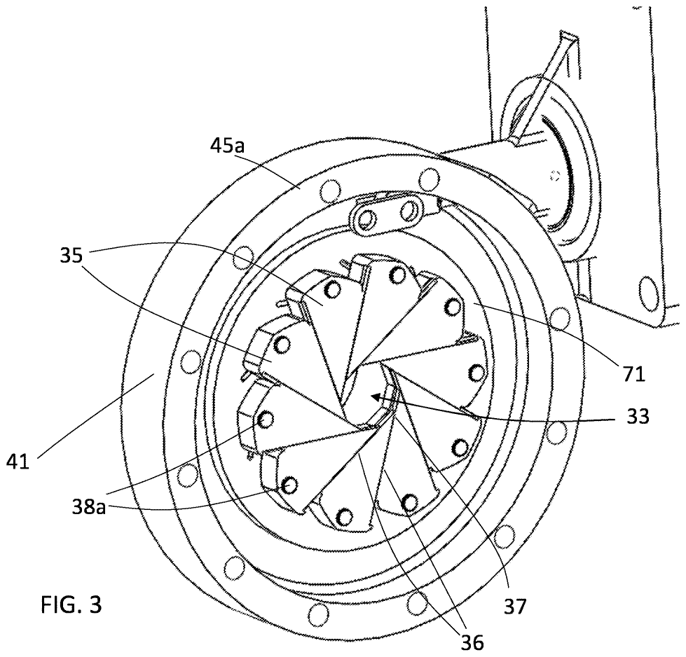

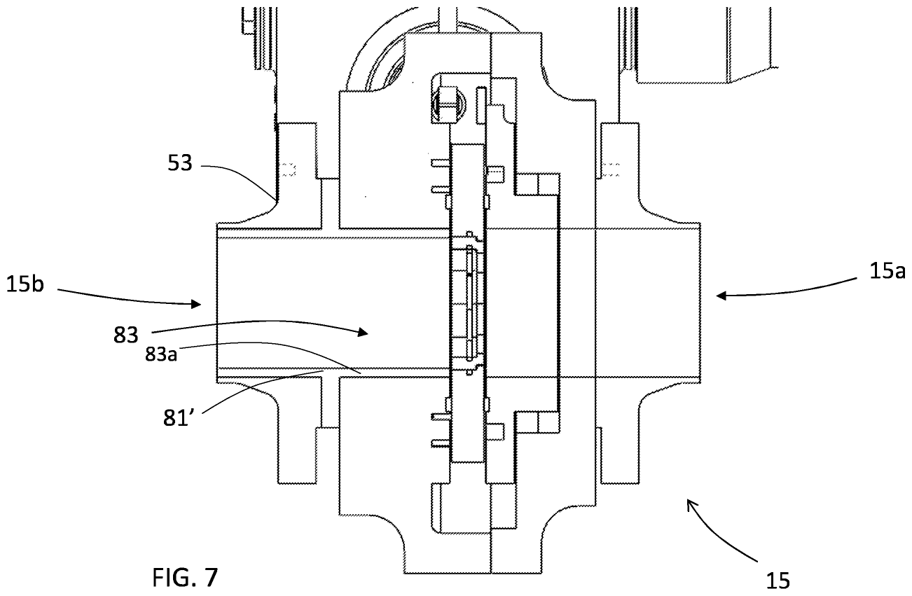

A blow valve 15 is shown in FIGS. 2-6. Different views and different parts of the blow valve 15 are shown in the different Figures. A blow valve 15 is shown in cross section in FIG. 7. A description of the blow valve 15 is now given with reference to all the Figures. The blow valve 15 comprises a shutter valve 31 with a central opening 33 of controllable size. The shutter valve 31 comprises a number of valve plates 35 (best seen in FIGS. 2 and 3) which are disposed in an annular pattern surrounding the central opening 33 of the shutter valve 31 and said valve plates 35 are movable in relation to each other whereby the size of the central opening 33 is controlled by moving the valve plates 35.

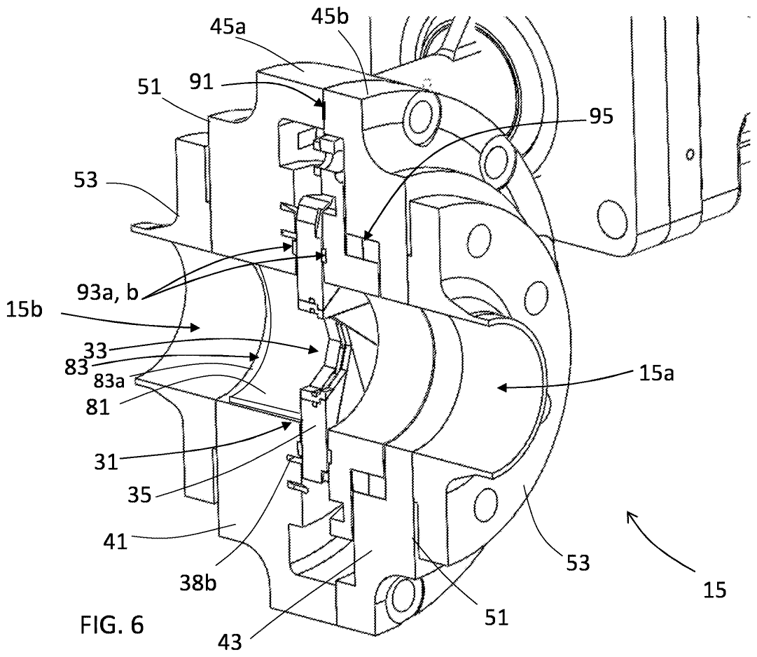

The blow valve 15 is designed with dimensions and materials for being usable and approved for pressures up to at least 25 bar. The different parts of the blow valve 15 need to be robust and wear resistance. The blow valve 15 comprises furthermore two flange connections 51 (seen in FIGS. 2 and 6) which are adapted for connection to flanges 53 (seen in FIG. 6) for connection to the blow line 7 and/or possibly one of them directly to the outlet 9 of the first vessel 3. The flange 53 and flange connection 51 are adapted for pressures up to at least 25 bar. For example, the flange 53 and flange connection 51 can be adapted for PN 40.

The blow valve 15 is configured for use in a biomass processing system being a biomass conversion system, for example a biomass conversion system for production of biogas, ethanol, biochar or biochemicals. As discussed above erosion is an even larger problem in biomass conversion systems because of the higher pressures, higher velocities, lower moisture content in the biomass material and possibly more wear producing particles such as sand in the material.

The blow valve 15 comprises a valve housing 41 and a valve cover 43 which together enclose the shutter valve 31. The valve housing 41 and the valve cover 43 comprise each a flange connection 51 for connection to the blow line 7 and/or the outlet 9 of the first vessel 3 as described above. The valve housing 41 and the valve cover 43 comprises each a peripheral connection flange 45a, 45b whereby the valve housing 41 and the valve cover 43 are configured for being connected to each other by mating their respective peripheral connection flanges 45a, 45b to each other and connect them. Between the two peripheral connection flanges 45a, 45b a first sealing 91 is provided for preventing leakage from the blow valve, which will be further described below.

The shutter valve 31 comprises between 6 and 16 or between 4 and 20 valve plates 35. In FIGS. 2-7, the shutter valve 31 comprises 10 valve plates 35, however any number between for example 6 and 16 or between 4 and 20 is suitable for the blow valve 15. The valve plates 35 have a thickness between 10-40 mm or between 15-30 mm. Hereby they are robust and can withstand pressures needed for this application. The valve plates 35 can be made from a resistant material having a high wear resistance, for example a high yield steel material. The material can be for example duplex stainless steel or super-duplex stainless steel. The valve plates 35 may further in some embodiments be coated with a wear resistant surface, such as a wear resistant lining. In the embodiments shown in FIGS. 2-7, the valve plates 35 have a form of a wedge having two straight side edges 36 which meet in a tip 37 of the wedge (best seen in FIG. 3). A movement pattern for the valve plates 35 for varying the size of the central opening 33 is, thanks to the straight side edges 36, relatively simple. Hereby the construction is robust and can withstand high pressures.

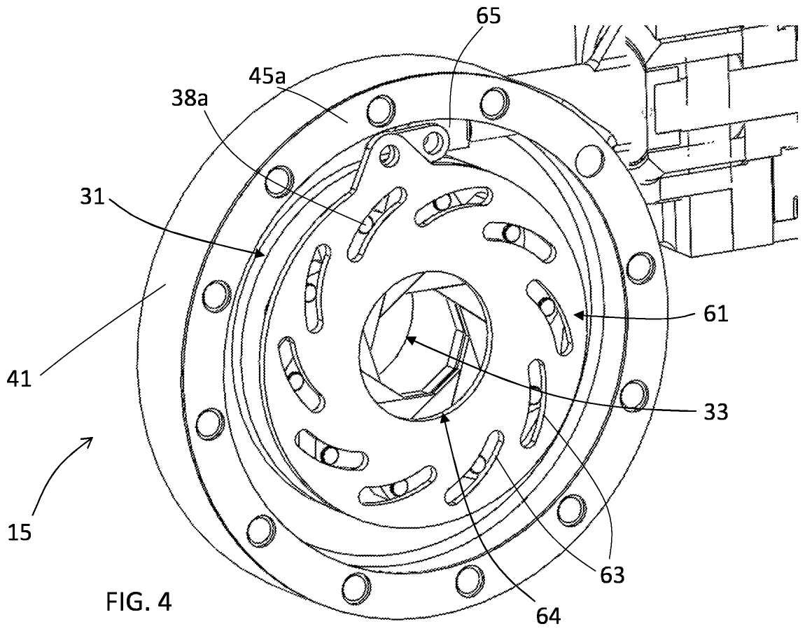

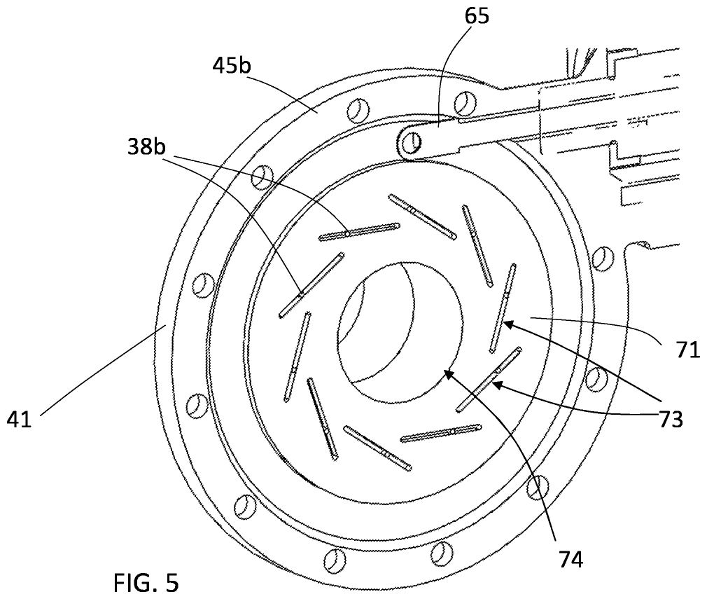

The construction for moving the valve plates 35 in relation to each other in order to control a size of the central opening 33 will now be briefly described with reference to FIGS. 1-6. A valve plate movement control disc 61 (best seen in FIG. 4) is provided in the shutter valve 31. The valve plate movement control disc 61 comprises a number of trails 63, which number corresponds to the number of valve plates 35. The trails 63 are in this embodiment curved trails distributed around a central opening 64 of the valve plate movement control disc 61. Each valve plate 35 comprises a first guiding pin 38a which fits into the trails 63 and one first guiding pin 38a is positioned in each trail 63. The valve plate movement control disc 61 is controlled by a rod 65 to rotate and when the valve plate moving control disc 61 is rotated the valve plates 35 are moved in relation to each other thanks to the first guiding pins 38a are forced in a direction allowed by the trails 63. On the opposite side of the valve plates 35 within the shutter valve 31 a number of straight trails 73 are provided. The straight trails 73 are provided in an inner surface 71 of the valve housing 41 (best seen in FIG. 5), which inner surface 71 and straight trails 73 are facing the valve plates 35. The number of straight trails 73 correspond to the number of valve plates 35. The straight trails 73 in the valve housing 41 are distributed around a central opening 74 of the valve housing 41. Each valve plate 35 comprises a second guiding pin 38b which second guiding pin 38b is positioned on an opposite side of the valve plate 35 compared to the first guiding pin 38a. One second guiding pin 38b is positioned in each of the straight trails 73. Hereby the movement of the valve plates 35 are further restricted according to the extension of the straight trails 73 and hereby the size of the central opening 33 is controlled by moving the rod 65 back and forth which in turn rotates the valve plate movement control disc 61 back and forth.

As best seen in FIG. 6 and FIG. 7, the blow valve 15 may further comprise a wear lining 81; 81′ which may be interchangeable. The wear lining 81; 81′ is positioned to cover an internal surface 83a of an outlet section 83 of the blow valve 15, which outlet section 83 is provided between the valve plates 35 and the outlet end 15b of the blow valve 15. The wear lining 81 can be designed as a tube section which can be changed when needed, as shown in FIG. 6. In FIG. 7, the wear lining 81′ is also covering an internal surface of the flange 53 and the wear lining 81′ comprises in this embodiment also an extending mounting flange 82 which is configured to be clamped between the flange 53 and the valve housing 41. Hereby the wear lining 81′ is kept in position and can also easily be changed. The wear lining 81; 81′ can be made from a suitable wear resistance material, such as for example high yield steel or a ceramic material. By using a wear lining 81; 81′ the blow valve 15 can be used a longer time before being destroyed by erosion. Furthermore, the wear lining 81; 81′ can be changed for a new wear lining 81; 81′ whereby life time of the blow valve can be extended even more.

Suitably the blow valve 15 comprises at least one sealing 91, 93a, 93b, 95 positioned in the blow valve such that leakage of biomass material and fluid via the blow valve outside said central opening 33 is limited.

The at least one sealing of the blow valve 15 comprises in some embodiments at least a first sealing 91 (seen in FIG. 6) positioned in the blow valve 15 to keep the pressure inside the blow valve 15. The first sealing 91 can be positioned between the peripheral connection flanges 45a, 45b of the valve housing 41 and the valve cover 43 when connected to each other. Hereby the first sealing 91 encircles all the valve plates 35 and prevents leakage between the valve housing 41 and the valve cover 43.

Two valve plate sealings 93a, 93b can furthermore in some embodiments be provided inside the blow valve 15. These valve plate sealings 93a, 93b are provided for limiting leakage of biomass material into the inner parts of the blow valve 15 but are also provided for allowing movement of the valve plates 15. The valve plate sealings 93a, 93b can for example be made from a highly wear-resistant UHMW-PE based material. The valve plate sealings 93a, 93b are circular and each of the valve plate sealings 93a, 93b is sealing towards all the valve plates 35. One valve plate sealing 93a is sealing towards one side of the valve plates 35 and the other valve plate sealing 93b is sealing towards the opposite side of the valve plates 35.

A second sealing 95 may furthermore be provided in some embodiments. The second sealing 95 is circular and is sealing between the valve cover 43 and the valve plate movement control disc 61. The second sealing 95 may be a stuffing box.

The blow valve 15 may be dimensioned to fit in a blow line 7 having a diameter between 50-150 mm.

According to the invention, a biomass processing system 1 for treatment of biomass material, as shown in FIG. 1, is provided. The biomass processing system 1 comprises a first vessel 3 in which said biomass material is treated under a first pressure, a second vessel 5 in which said biomass material is received and held at a second pressure which is lower than the first pressure. Often the second pressure is atmospheric pressure as discussed above. The biomass processing system 1 comprises furthermore a blow line 7 connecting an outlet 9 of the first vessel 3 with an inlet 11 of the second vessel 5 for transporting the biomass material from the first vessel to the second vessel and a blow valve as described above. The blow valve is connected in said blow line 7 and is configured for controlling the flow of biomass material and fluid in the blow line 7.

The difference between the first and second pressures can for example be between 10-25 bar. The blow valve is, as described above, suitably designed with dimensions and materials for being usable and approved for pressures up to at least 25 bar.

The biomass processing system 1 is in some embodiments of the invention a biomass conversion system for production of for example biogas, ethanol, biochar or biochemicals.

In one embodiment of the invention the blow valve 15 is connected directly to the outlet 9 of the first vessel 3. In one embodiment of the invention the blow line 7 has a diameter between 50-150 mm.

Further, a vessel 3 for use in a biomass processing system 1 may be provided, wherein said vessel 3 is a pressurized vessel for processing of biomass and comprises a blow valve 15 as described above, which blow valve is connected to an outlet 9 of the vessel 3. Said vessel 3 may be a reactor in a biomass conversion system.

1.-11. (canceled)

12. A biomass processing system for treatment of biomass material, wherein said biomass processing system is a biomass conversion system, said biomass processing system comprising:
a first vessel configured to hold said biomass material under a first pressure during treatment of the biomass material;
a second vessel configured to receive said biomass material and to hold said biomass material at a second pressure which is lower than the first pressure, wherein a difference between the first and second pressures is at least 10 bar;
a blow line connecting an outlet of the first vessel with an inlet of the second vessel for transporting the biomass material from the first vessel to the second vessel; and
a blow valve connected in said blow line and configured for controlling the flow of biomass material and fluid in the blow line and configured to control a steam explosion of the material occurring at the passage through the blow valve, wherein said blow valve comprises a shutter valve with a central opening of controllable size, said shutter valve comprising a number of valve plates which are disposed in an annular pattern surrounding the central opening of the shutter valve and wherein said valve plates are movable in relation to each other whereby the size of the central opening is controllable by moving the valve plates.

13. Biomass processing system according to claim 12, wherein the blow valve is designed with dimensions and materials for being usable and approved for pressures up to at least 25 bar(g).

14. Biomass processing system according to claim 12, wherein the shutter valve comprises between 4 and 20 valve plates.

15. Biomass processing system according to claim 12, wherein said valve plates have a thickness of between 10 mm and 40 mm.

16. Biomass processing system according to claim 12, wherein said valve plates are made from a high yield steel material.

17. Biomass processing system according to claim 12, wherein said valve plates are coated with a wear resistant lining.

18. Biomass processing system according to claim 12, wherein said valve plates have a form of a wedge having two straight side edges which meet in a tip of the wedge.

19. Biomass processing system according to claim 12, wherein the blow valve further comprises a wear lining.

20. Biomass processing system according to claim 12, wherein said blow valve comprises at least one sealing positioned in the blow valve such that leakage of biomass material and fluid via the blow valve outside said central opening is limited.

21. Biomass processing system according to claim 12, wherein the blow valve is connected directly to the outlet of the first vessel.

22. Biomass processing system according to claim 12, wherein the blow line has a diameter between 50-150 mm.
